(19) United States
(12) Patent Application Publication
(10) Pub. No.: US 2015/0065855 A1
(43) Pub. Date: Mar. 5, 2015

(54) CARDIAC IMAGING PROCESSING FOR INTERVENTIONS

(71) Applicant: UMC Utrecht Holding B.V., Utrecht (NL)

(72) Inventors: Frebus Jan VAN SLOCHTEREN, Utrecht (NL); Rene VAN ES, Utrecht (NL); Steven Anton Jozef CHAMULEAU, Utrecht (NL)

(73) Assignee: UMC Utrecht Holding B.V., Utrecht (NL)

(21) Appl. No.: 14/250,563

(22) Filed: Apr. 11, 2014

Related U.S. Application Data
(60) Provisional application No. 61/871,320, filed on Aug. 29, 2013.

(51) Int. Cl. A61B 5/00; A61B 19/00; A61B 10/02; A61B 5/055; A61M 25/00

(52) U.S. Cl. CPC A61B 5/0035; A61B 5/055; A61M 25/0084; A61B 10/0233; A61B 19/5225
USPC 600/411; 600/407; 604/510; 600/562

(57) ABSTRACT

A method of processing an image includes determining a local disease severity measure based on a 3D dataset. The method further includes determining a target region for a treatment, based on the local disease severity measure, wherein the target region corresponds to a transition region between a first region and a second region, wherein the first region has values of the local disease severity measure which are substantially distinct from the values of the local disease severity measure in the second region. The method further includes registering at least the target region with an interventional image modality. For example, the local disease severity measure is indicative of a measure of a local disease severity at a location along a surface of a myocardium.

### FIELD OF THE INVENTION

The invention relates to cardiac image processing for interventions.

### BACKGROUND OF THE INVENTION

Cardiac regenerative therapy for ischemic heart disease (IHD) aims to provide local cardiac protection and regeneration by means of vasculogenesis, cardiomyogenesis, and matrix support. Studies have shown that injection of stem/progenitor cells into the border zone of the infarcted area helped to stimulate cardiac repair via cell-to-cell contact and secretion of paracrine factors. Moreover, therapeutic effects may rely on the delivery and retention of the regenerative therapeutics on a location where oxygen and nutrients are available to enable survival. Via intramyocardial catheters stem cells, stem cell derived factors, progenitor cells, and/or biomaterials can be injected with minimal invasiveness into the myocardium. Injection locations can be chosen based on tissue viability measures obtained from electromechanical mapping (EMM). For example, NOGA®XP (Biosense Webster, Cordis, Johnson & Johnson, USA) is an intramyocardial injection system that provides a three dimensional (3D) magnetic tracking technology and allows for the assessment of local electrical and mechanical tissue characteristics. Using the injection system, local unipolar (unipolar voltage, UV) and bipolar (bipolar voltage, BV) depolarization potentials and relative catheter tip displacements (Linear Local Shortening, LLS) are measured at multiple locations on the left ventricular (LV) endocardium. These measurements are interpolated to obtain a three-dimensional reconstruction of the LV endocardium which is used to guide stem cell injections.

### SUMMARY OF THE INVENTION

Since the measures of electromechanical mapping are often not distinctive in non-transmural infarctions, and measurements are interpolated in regions where no measurements are taken, the results obtained by electromechanical mapping are not reproducible, and prone to errors. Moreover, since the non-transmural border zone of the infarction is believed to be the preferred delivery site of the stem cell therapeutics, it is crucial for it to be optimally defined during the injection procedure.

An aspect of the present invention is to provide a method for optimizing a cell delivery location by combining the gold standard measure of infarct transmurality or other infarct, ischemia (perfusion/viability), or myocardial deformation related parameters derived from MRI or other imaging modalities and practical guidance during operation.

Another aspect of the present invention is to provide a system for facilitating real-time image guided (pre-) clinical stem cell injection. This system provides the possibility to guide intramyocardial injections with high accuracy to locations with distinct infarct, ischemia (perfusion/viability), or myocardial deformation characteristics. In addition, this system can be used to specify the definition of border zone of the infarct.

In an aspect, the invention provides a method of processing an image, comprising

determining a local disease severity measure based on a 3D dataset;

determining a target region for a treatment, based on the local disease severity measure, wherein the target region corresponds to a transition region between a first region and a second region, wherein the first region has values of the local disease severity measure which are substantially distinct from the values of the local disease severity measure in the second region; and

registering at least the target region with an interventional image modality.

The system allows the target region for the treatment to be clearly defined in the interventional image modality, so that during an intervention, the target region can be reached more reliably.

The said local disease severity measure may be indicative of a measure of a local disease severity at a location along a surface of a myocardium. This allows for example an intravascular catheter to be guided through the ventricle towards the portion of the inner surface of the myocardium corresponding to the target region.

The local disease severity measure may be indicative of a transmurality of an infarction of a myocardium or of a local severity of ischemia. The transmurality may be a good indicator to identify the region where to perform an interventional treatment, in particular a stem cell injection. For example, a local signal intensity may be correlated with disease severity. Consequently, the signal intensity may be used as another local disease severity measure.

The transition zone may be a border zone of an infarcted region of the myocardium. Such a region may be particularly suited for a stem cell injections in clinical practice.

The determining the target region may comprise comparing the local disease severity measure to a reference value. Such a reference value may be used to locate the most viable region for the intervention.

The target region may comprise a region where the local disease severity is equal to the reference value, a region where the local disease severity is smaller than the reference value, and a region where the local disease severity is larger than the reference value. The target region may thus contain a border zone around a region having the reference value.

The target region may correspond to a region comprising an isoline where the local disease severity is equal to the reference value and a margin on both sides of the isoline. It has been found that such a margin around an isoline provides relatively good results.

The margin may have a predetermined width measured along a surface of the myocardium, on both sides of the isoline. It has been found that such a margin around an isoline provides relatively good results. The width may be, for example, around 5 mm on each side of the isoline. However, this is not a limitation.

The method may comprise determining a no-go zone for intervention corresponding to an area with a high local disease measure according to a set of predetermined constraints. Clear identification of such a no-go zone provides additional safety for the patient, because an injection in the highly diseased area, for example a core infarction area, may impose a risk to the patient.

The 3D dataset may comprise one or a combination of a nuclear magnetic resonance (NMR) dataset, a magnetic resonance imaging (MRI) dataset, a computed tomography (CT) dataset, a PET dataset, a SPECT dataset, an echography dataset, and a spectroscopy dataset. These types of images may be suitable for determining the local disease severity. For example, a fusion of MRI and SPECT may be advantageously used.

The 3D dataset may comprise an MRI image, and the MRI image may comprise a late gadolinium enhanced image. This type of image may be particularly useful to determine local disease intensity, for example in terms of transmurality or signal intensity.

The interventional image modality may comprise an interventional navigation system. Such an interventional navigation system may be used to guide a catheter tip inside the heart, for example inside the left ventricle, towards a portion of the myocardium, based on the identified target region for treatment.

The interventional image modality may comprise a magnetic resonance imaging modality. Also, the method may comprise acquiring a magnetic resonance imaging dataset while navigating the catheter inside a body. The step of registering may comprise registering the target region with the magnetic resonance imaging dataset that was acquired while navigating the catheter inside the body. This allows the intervention to be guided by means of MRI, while using the target region to guide the intervention.

The method may comprise displaying the registered target region fused with an image of the interventional image modality during an intervention. The image of the interventional image modality during the intervention may, for example, be indicative of a location of an interventional instrument, and/or real-time anatomical information related to an interventional instrument, and the fused image containing both the target region and the image of the interventional image modality may help to locate the target region with the instrument and/or in respect of the real-time anatomical information.

The method may comprise displaying the registered target region in a bull's eye plot format or a polar coordinate system. Such a format may facilitate the understanding of the target region by an observer.

The method may comprise directing a catheter to the target region. This may be performed automatically, by means of an interventional guiding mechanism. This step may alternatively be performed manually by an interventionalist.

The method may further comprise injecting at least one stem cell, or stem cell derived factors, or medication, or biomaterials, or a combination thereof, into the target region using the catheter.

The method may further comprise performing an ablation or performing a biopsy in the target area using the catheter. This may be another application of the invention.

In another aspect, the invention provides a system for processing an image, comprising

a disease severity measurement determining unit for determining a local disease severity measure based on a 3D dataset;

a target region determining unit for determining a target region for a treatment, based on the local disease severity measure wherein the target region corresponds to a transition region between a region with a low local disease severity measure and a region with a high local disease severity measure;

a registering unit for registering at least the target region with an interventional image modality.

The system may comprise a guiding unit for controlling an intramyocardial catheter guiding apparatus to guide the catheter towards the target region based on the registered target region.

The system may comprise means to specify the definition of border zone of the diseased location. For example, the system may comprise a user input unit for receiving input regarding the definition of border zone. The definition of border zone may comprise, as a non-limiting example, a particular disease severity level corresponding to the border of an infarction area, and a particular width of the border zone.

In another aspect, the invention provides a system for use in real-time image guided stem cell injection, the system comprising a guiding unit for controlling an intramyocardial catheter guiding apparatus to guide an intramyocardial catheter to a location on a target region corresponding to a border zone of a diseased area according to infarct transmurality, ischemia, or myocardial deformation.

The system may comprise a disease severity measurement determining unit for determining a local disease severity measure based on a 3D dataset.

The system may further comprise a target region determining unit for determining the target region, based on the local disease severity measure, wherein the target region corresponds to a transition region between a region with a low local disease severity measure and a region with a high local disease severity measure, in accordance with a set of constraints.

The person skilled in the art will understand that the features described above may be combined in any way deemed useful. Moreover, modifications and variations described in respect of the system may likewise be applied to the method and to the computer program product, and modifications and variations described in respect of the method may likewise be applied to the system and to the computer program product. The person skilled in the art will understand that many variations are possible, without departing from the spirit and scope of the appended claims, including equivalents.

### BRIEF DESCRIPTION OF THE DRAWINGS

In the following, aspects of the invention will be elucidated by means of examples, with reference to the drawings. The drawings are diagrammatic and may not be drawn to scale.

FIG. 1 is a sketch of a system for guiding an interventional catheter.

FIG. 2 is a flow chart of a method of cardiac image acquisition and processing for an intervention.

FIG. 3 is a diagram illustrating the concept of transmurality.

FIG. 4 is a bull's eye plot showing a target area for an intervention.

FIG. 5 is a visualization of a 3D mesh showing a target area for an intervention, overlaid on a medical image.

FIG. 6 is a flowchart of a method of cardiac image processing for an intervention.

FIG. 7 is a block diagram of a system for cardiac image processing for an intervention.

### DETAILED DESCRIPTION OF EMBODIMENTS

In the following description, a number of embodiments of the invention will be described in more detail. However, it should be noted that the detailed description presents example embodiments, which do not limit the invention.

According to a method of treatment planning in cardiac stem cell therapy, prior to the injection procedure, gold standard diagnostic imaging techniques are used to assess the severity of the disease. Using MRI, infarct transmurality may be assessed using late gadolinium enhancement. Perfusion of the myocardium may be assessed. Deformation of the myocardium may be assessed, for example using MRI tagging. Using SPECT/CT, perfusion and/or viability of the myocardium may be assessed. These are examples of local disease severity measures. The diagnostic imaging data may be converted into a target area by an automatic algorithm using user defined thresholds for the local disease severity measure. For an image guided injection, the target area may be projected on a 3D endocardial mesh. The mesh may be co-registered with an electromechanical mapping (EMM) catheter. Other catheter injection systems may also be used. The target area (or areas) may be used to guide the injections. A good visual agreement of the infarct border zone may be obtained by using an infarct transmurality threshold of 50%, and an area width for the target area of 1 cm. However, the threshold and the area width are not limited to the values of 50% and 1 cm. These values may be modified as needed. For example, it is possible to target areas and border zones at any percentage between 0 and 100% transmurality. Other parameter settings may also provide good results. These parameters can be adjusted by the user. The method enables assignment of an optimal injection area based on the gold standard diagnostic imaging data, and precise injections into this area. The solution may provide one or more of the following advantages: gold standard diagnostic imaging, automated target area assignment, standardized injection strategies, easy image guided injection procedures, and standardization of injection strategies.

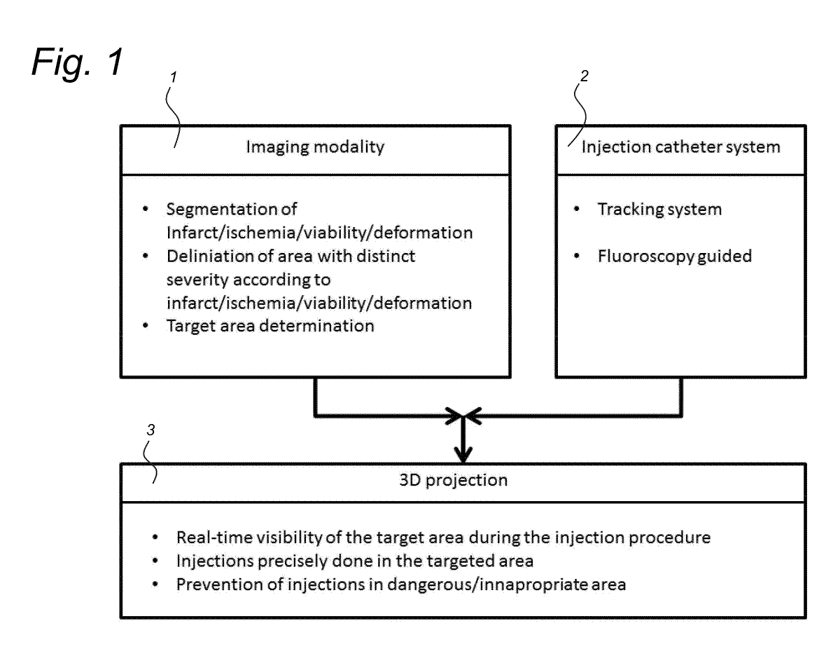

FIG. 1 illustrates a system for guiding an interventional catheter. Generally, three components may be distinguished. The first component 1 represents one or more imaging modalities which may be used to obtain an image of an anatomy. The obtained image data may be used to make a diagnosis and/or decide on a treatment. Moreover, the obtained image data may be processed by the system, to identify a target area for cardiac regenerative therapy, for example. To this end, certain processing operations may be performed as described hereinafter in more detail. For example, the processing may comprise a segmentation based on one or more severity threshold values. The second component 2 represents a catheter tracking system. Such a system may use a trackable catheter, for example using a tracking technology such as magnetic or impedance measurements to localize at least part of a catheter. Alternatively, the catheter tracking system may be based on image detection, i.e. by means of an image guided catheter system. For example, the catheter tracking may be guided using MRI data that is acquired during the intervention. This way, MRI-based interventional modality may be provided. Alternatively, the catheter tracking might be guided using fluoroscopy. The catheter system may be configured to perform injections of a myocardium, for example, under control of automated and/or manual catheter guidance means. The third component 3 represents a subsystem which is configured to combine the target area identified by the first component 1 and the catheter. That is, the target area may be combined with a catheter position determined by the second component 2, for example. Moreover, image data acquired by the second component 2 during catheter manipulations may be combined with the target area, for example to depict the target area in the context of the image data acquired by the second component. This way the image guided injections may be guided towards the target region.

Hereinafter, embodiments of the invention will be described with reference to a novel software toolbox (“3D CartBox”), which was developed by the inventors. The software toolbox is capable of using the NOGA®XP system and MRI data, in order to provide real-time image guidance for intramyocardial stem cell injections.

The result of cardiac regenerative therapy importantly depends on optimal delivery of biologics to the myocardium. It was found that it may be beneficial if intramyocardial catheter guided stem cell transplantations are performed in border zone areas. Such border areas may be the closest regions of viable (thus perfused) myocardium in the vicinity of the infarct area. The gold standard technique to determine this area is late gadolinium enhanced magnetic resonance imaging (LGE-MRI). Myocardial viability assessment by the NOGA®XP electromechanical mapping (EMM) catheter guided injection system is prone to errors in non transmural infarctions.

In a preferred method, LGE-MRI and EMM data are obtained. MRI and EMM datasets may be registered using a suitable registration algorithm. Several registration algorithms are known in the art per se. In an embodiment, the registration may be performed by any one or more of the following three steps: 1) landmark registration, 2) surface registration, and 3) manual optimization. Examples of landmarks which may be used for the registration include the apex and the coronary ostia After registration, the EMM data may be projected on an endocardial surface mesh derived from MRI.

The registration algorithm may enable registration of the EMM on pre-acquired MRI during an injection procedure. This may be useful to visualize the complex 3D scar geometry, and allows the operator to perform dedicated, real-time cell injections that are more accurately guided to the border zone of the infarct.

Accurate identification of viable tissue in proximity of the infarct is of great importance. Via intramyocardial injection catheters stem/progenitor cells or biomaterials can be injected with minimal invasiveness into the myocardium. Moreover, since the non transmural border zone of the infarction is believed to be the preferred delivery site of the stem cell therapeutics [2], it is crucial for it to by optimally defined during the injection procedure. In this disclosure, a practical toolbox (3D CartBox) is described that allows real-time image guided (pre-) clinical stem cell injection. This toolbox provides the possibility to guide intramyocardial injections with high accuracy to locations with a distinct infarct transmurality.

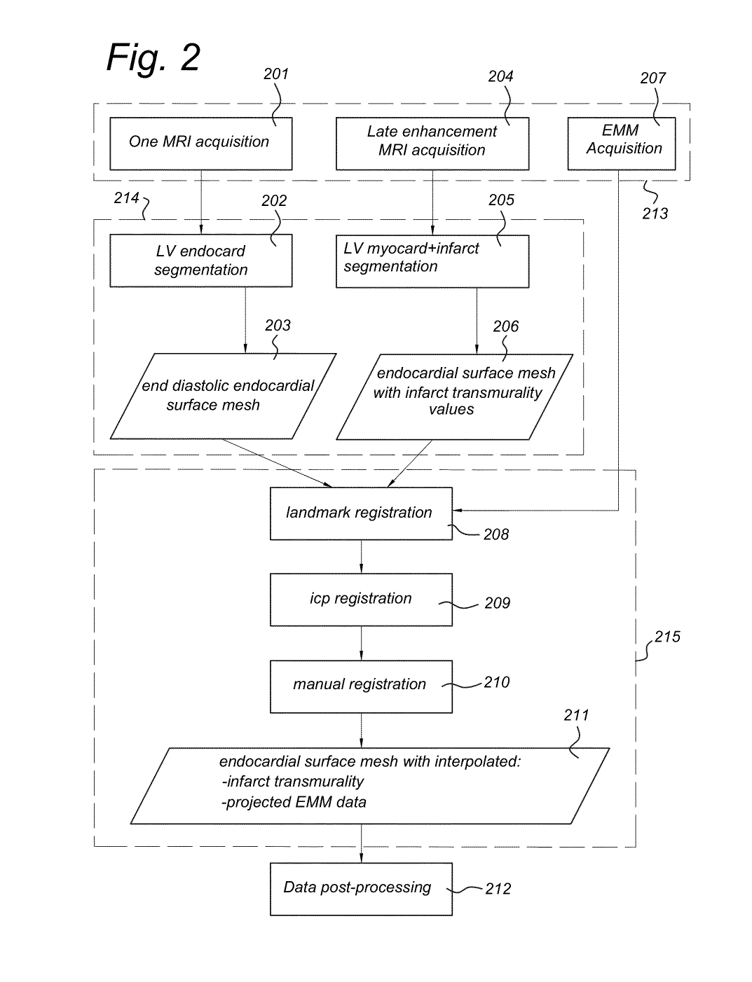

FIG. 2 illustrates an example of the 3D CartBox process. The process can be sub-divided into four major steps: Data acquisition 213, Data pre-processing 214, Registration 215, Post processing 212. The acquisition 213 can comprise an optional cine MRI acquisition 201, a late enhancement MRI acquisition 204, and an EM Map acquisition 207.

Although FIG. 2 illustrates the use of MRI acquisitions as the prior image dataset used to determine the target zone, this is only an example. The MRI acquisitions may be replaced and/or complemented with 3D dataset from another modality, such as SPECT/CR or echography, or any other suitable imaging modality.

The data pre-processing step 214 may comprise LV endocard segmentation 202 of the cine MRI dataset, resulting in end diastolic endocardial surface mesh 203. The data processing step 214 may further comprise LV myocard and infarct segmentation 205, which results in an endocardial surface mesh with infarct transmurality values 206.

The registration step 215 may comprise landmark registration 208, iterative closest point (ICP) registration 209, and/or manual registration 210. As mentioned above, any kind of surface registration may be applied, for example to replace the ICP registration 209. The registration step 215 may result in an endocardial surface mesh 211 with interpolated infarct transmurality and projected EMM data.

The data post-processing step 212 may further process the endocardial surface mesh 211. Such data post-processing 212 may include, for example, calculation of match/mismatch between LGE-MRI and EMM parameters, and/or visualization of bullseye plots.

The NOGA® XP system (Biosense Webster, Cordis, Johnson & Johnson, USA) version 1.1.43 may be used equipped with a 7 French NOGA mapping catheter (Biosense Webster, Cordis, Johnson & Johnson, Diamond Bar, USA) for the mapping procedure. However, this is only an example of an interventional EMM system. Other catheter injection systems can also be used.

In general, the LV may be entered by retrograde passage through the aortic valve. Readout of the catheter tip location may be done in end-diastole using R-wave triggering. Thereby providing, for example, only end-diastolic locations for registration. Points taken in the left and right coronary ostia and the apex may serve as anatomical landmarks. The apex location may be taken as the most outward point that is reached in the apical region confirmed by fluoroscopy. To obtain data from the interventional system in a real time fashion, the system may be configured to enable, for example, read-only access from an external computer running, for example, 3D CartBox, as disclosed herein.

Data Pre-Processing

The coronary ostia, apex may be selected manually in the end-diastolic frames of the cine images. Segmentation of the endocardium may be done in the end-diastolic frames of the short axis cine images to create a 3D surface mesh of the left ventricular endocardium for surface registration and projection of the acquired data. Segmentations may be performed using existing post processing software for medical images. However, this is not a limitation. Endocardial segmentation may be done automatically and corrected manually if necessary. In the short axis LGE datasets the endo-, and epicardial contours and the MI contours may be segmented automatically and corrected manually if necessary e.g. by an experienced physician. Infarct size and transmurality may be quantified using an existing segmentation algorithm. The short axis datasets may be divided in 80 radial sectors and area based infarct transmurality assessment may be done using a modified bullseye function of Segment. Infarct transmurality data may be projected on the 3D surface mesh obtained from the cine images for comparison to the EMM data.

Perioperative Registration

For perioperative registration the raw data obtained from the NOGA®XP system may be used. The raw NOGA®XP dataset and the MRI datasets may be registered coarsely based on the anatomical landmarks, for initial registration using a closed-form least squares approach [18, 19]. This algorithm may be modified, for example to enable weighing the contribution of the locations used for landmark registration. Alternatively, other forms of registration may be applied. Hereafter the EMM catheter location and orientation may be visualized in the MR images during the catheter procedure. Optionally, after acquiring points in all or several regions of the left ventricular endocardium, it is possible to optimize the registration using, for example, an iterative closest point (ICP) algorithm [19]. The ICP algorithm may be restricted to small rotations around the apex in the sagittal, coronal, and transverse plane of respectively 10, 20 and 20 degrees, for example, to prevent excess rotations. If necessary the registration may be manually optimized by manually adjusting the location and orientation interactively with six degrees of freedom if so desired. Optionally, the accuracy of the registration may be calculated, for example as the mean distance from the EMM points to the mesh surface as previously described [20].

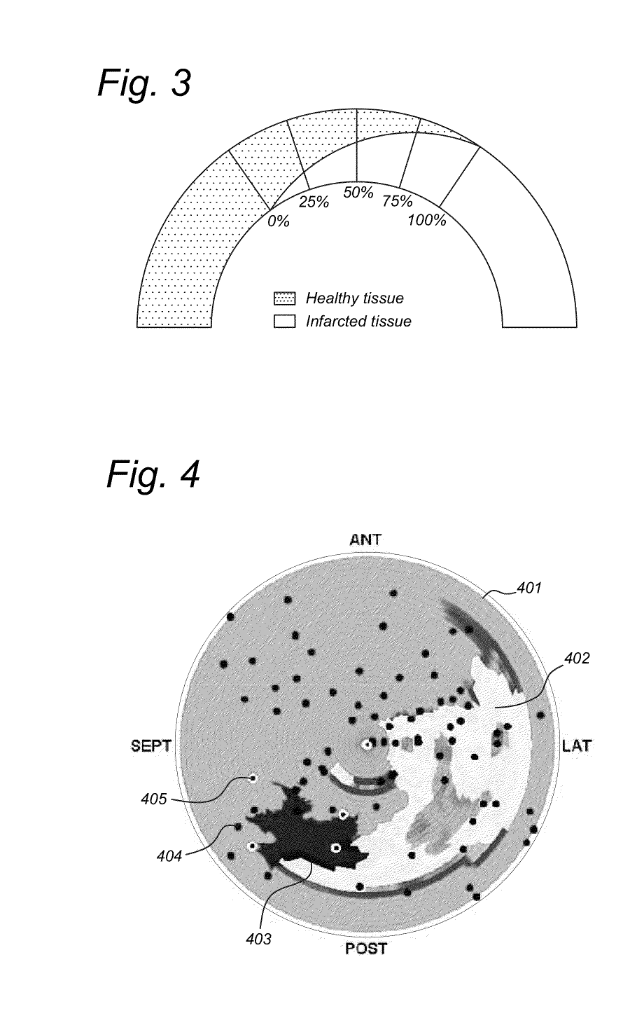

The endocardial surface may be subdivided in four sections with a different infarct transmurality as schematically shown in FIG. 3A. Based on the rationale that 100% transmurally infarcted myocardium most likely is not a location where the stem cells are supplied with sufficient oxygen and nutrients [2], and at regions with 0% transmurality there is no use for stem cells, one may refer to the 50% infarct transmurality area as viable tissue applicable for stem cell therapy. Moreover, since the first evidence of cardiac regeneration was shown in the border zone, this may be applied as a target area [2].

Clinical Implications

Incorrect injections of stem cells into the myocardium might importantly restrain the success of cardiac regenerative therapy [7]. Therefore a technique that can accurately identify the border zone is of great importance. The use of the gold standard fibrosis imaging technique could facilitate more accurate characterization of the complex 3D scar geometry and thereby optimally guide injections to the infarct border zone. Consequently this could lead to shorter injection procedures, less necessity for the use of fluoroscopy to confirm the injection location, and less radiation for the patient and the physician. Therefore real-time integration of LGE-MRI during cardiac stem cell injection procedures could be a key to harnessing the full therapeutic effects of cardiac stem cell therapy. With the techniques disclosed herein, the parameters (such as perfusion, fibrosis, myocardial tissue tagging) from a pre-procedural acquired MRI or other imaging modality (SPECT/CT) may be used to guide the stem cell injection procedures. The techniques disclosed herein may make use of standard MRI pre-processing techniques to drive routine use in daily clinical practice, and ultimately helps to improve cardiac function to a larger extent, and increase the patient's longevity.

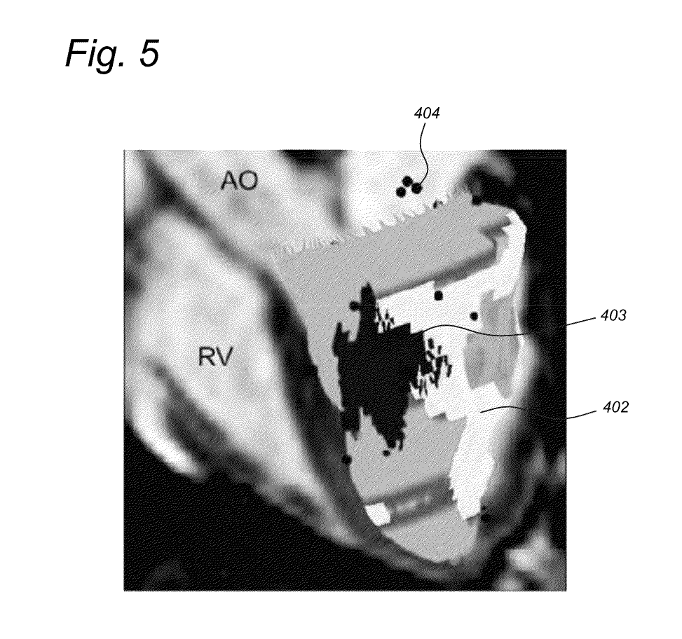

FIG. 4 illustrates a bull's eye plot 401 of a border zone 402 of an infarcted area. FIG. 5 illustrates a 3-chamber view with mesh. The same reference numerals have been used for the similar items in these figures. The border zone 402 is a 1 cm wide border zone with 50% infarct transmurality. Black dots 404 are the NOGA measurement points, white circled dots 405 are 4 injection locations+the apex position. ANT means anterior, POST means posterior, SEPT means septal, LAT means lateral. RV=right ventricle, AO=Aorta. The figure also shows an area 403 which is the infarct border zone as targeted by the NOGA parameters: UV>7 mV and LLS<=6%.

Using the techniques disclosed herein, it is possible to perform real time registration of EMM and MRI data that combines the high accuracy of the cardiac navigation with detailed information obtained from MRI during cardiac stem cell injection procedures. In future applications these fused images can be used to optimize the injection locations further.

The present invention is not limited to the embodiments described, but extends also to other embodiments without departing from the scope and spirit of the invention as defined by the appended claims, and their equivalents.

A method to provide guidance of intramyocardial catheters during cardiac regenerative therapy for ischemic heart disease to delivery locations, may comprise the following steps of:

measuring of disease severity with previously obtained 3D cardiac image;

generating target areas based on the infarct transmurality, ischemia (tissue perfusion or viability) or myocardial deformation; and

rendering the target areas in the navigation system during operation.

The method may use 3D cardiac images derived from MRI, CT, PET, SPECT or Echo, or a combination of MRI and SPECT, for example.

Moreover, it is possible to perform specific rendering of no-go areas with high disease severity/thin myocardium, for example.

Moreover, it is possible to render the target areas in a polar distribution format.

Moreover, it is possible to perform automatic registration of the obtained 3D cardiac image to the navigation system.

Moreover, it is possible to perform a step of receiving user input to provide characteristics to define the target areas.

A system may be implemented to provide real-time image guided (pre-) clinical stem cell injection comprising means to guide an intramyocardial catheter to locations with a distinct infarct transmurality, ischemia (tissue perfusion or viability) or myocardial deformation. The system may comprise means to specify the definition of a border zone of the diseased location.

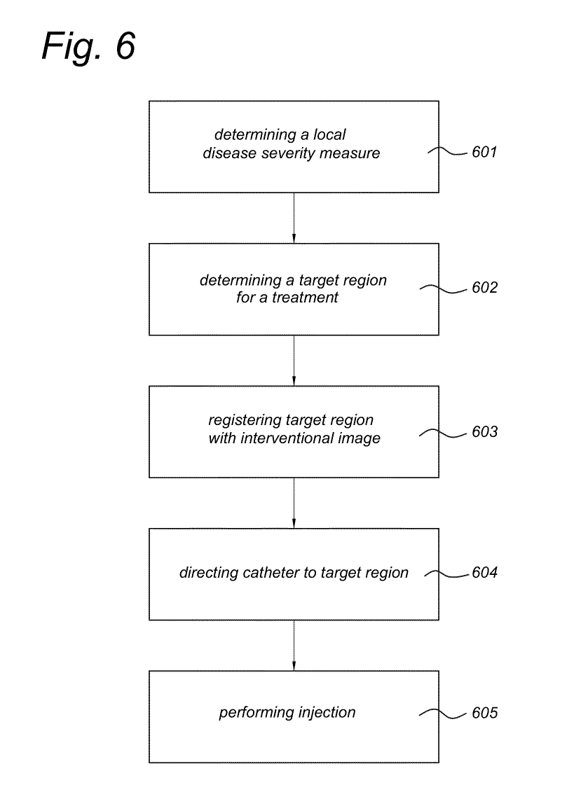

FIG. 6 illustrates a method of processing an image. The method comprises step 601 of determining a local disease severity measure based on a 3D dataset. Further, the method comprises step 602 of determining a target region for a treatment. Step 602 may be based on the local disease severity measure, wherein the target region corresponds to a transition region between a first region and a second region, wherein the first region has values of the local disease severity measure which are substantially distinct from the values of the local disease severity measure in the second region. An example of disease serverity measure is transmurality of an infarct of the myocardium. Another example may be a signal intensity of a properly measured signal. Further, the method comprises step 603 of registering at least the target region with an interventional image modality. More regions may be determined and registered, such as a 100% infarcted region and/or a healthy region. Moreover, the MRI dataset may be registered with the interventional image modality. For example, the interventional image modality is an image modality that is operative to be used during an electromechanical mapping procedure or a fluoroscopy guided interventional imaging modality.

The local disease severity measure may be indicative of a measure of a local disease severity at a location along a surface of a myocardium. For example, the values relate to an inner surface of the myocardium, where the catheter may puncture the inside of the heart walls.

For example, the local disease severity measure is indicative of a transmurality of an infarction of a myocardium or of a local severity of ischemia. The transition zone may be a border zone of an infarcted region of the myocardium. Step 602 of determining the target region may comprise comparing the local disease severity measure to a reference value. The target region may comprise a region where the local disease severity is equal to the reference value, a region where the local disease severity is smaller than the reference value, and a region where the local disease severity is larger than the reference value.

The target region may correspond to a region comprising an isoline where the local disease severity is equal to the reference value and a margin on both sides of the isoline. That margin may have a predetermined width measured along a surface of the myocardium, on both sides of the isoline.

Besides the target region, the method may comprise a step of determining a no-go zone for intervention corresponding to an area with a high local disease measure according to a set of predetermined constraints. The high local disease measure may be indicative of fully infarcted tissue.

The 3D dataset may comprise one or a combination of an nuclear magnetic resonance dataset, a magnetic resonance imaging dataset, a late gadolinium enhanced magnetic resonance imaging dataset, a computed tomography dataset, a PET dataset, a SPECT dataset, an echography dataset, and a spectroscopy dataset. The 3D dataset may be a non-interventional image dataset. Moreover, the 3D dataset may be a volume dataset comprising voxels representing intensity values of a measured parameter in three dimensions. As known in the art, a volume dataset may comprise a plurality of slices, each slice comprising a plurality of voxels.

The method of any preceding claim, wherein the interventional image modality may comprise an interventional navigation system. The method may comprise displaying the registered target region fused with an image of the interventional image modality during an intervention. For example, the method comprises displaying the registered target region in a bull's eye plot format or a polar coordinate system.

The interventional image modality may be configured to generate fluoroscopy images, such as x-ray images and/or surface data obtained from electromechanical mapping.

The method may further comprise step 604 of directing a catheter onto the target region. This step may be performed using the guiding technology of the EMM interventional device that is used. NOGA® XP and Carto are examples of such devices. However, the catheter may also be directed manually to the target region by a clinician, using the fused displayed target region and catheter position.

The may further comprise step 605 of injecting at least one stem cell, or stem cell derived factors, or medication, or biomaterials, or a combination thereof, into the target region using the catheter. After the catheter has been guided into the target region, the stem cells may be injected at the selected location or locations within the target region.

Alternatively, step 605 of injecting a stem cell may be replaced by a step of performing an ablation or performing a biopsy in the target area using the catheter.

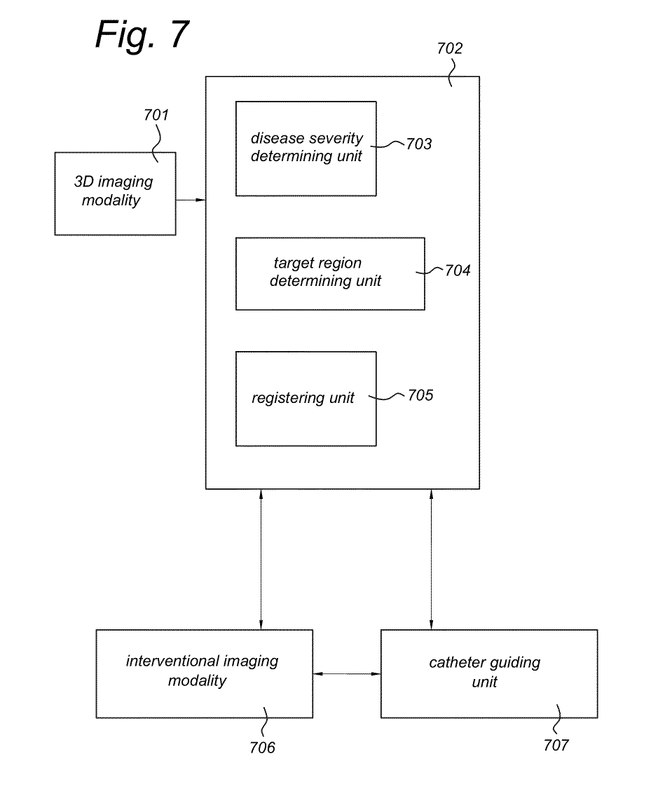

FIG. 7 illustrates a system comprising one or more apparatuses enabling a clinical intervention. The system may comprise a data processing unit 702. Parts of the system, in particular the data processing unit 702, may be implemented by means of a suitably programmed computer. The system may also be implemented by means of dedicated electronic circuitry. Moreover, the system may be configured to communicate with a 3D imaging modality 701 (such as MRI, but this is not a limitation), and an interventional imaging modality 706 and/or a catheter guiding unit 707 The system 702 may also be embedded in either of the 3D imaging modality 701, the interventional imaging modality 706, and/or the catheter guiding unit 707. The interventional imaging modality 706 and the catheter guiding unit 707 may be an integrated apparatus or may be separate systems.

In an embodiment, the interventional imaging modality 706 comprises an MRI apparatus, configured to acquire MRI datasets at one or more time points during the intervention. For this purpose, the catheter may be an MRI compatible catheter. It is possible that both the 3D dataset acquired before the intervention, on the basis of which the target region is determined, and the data from the interventional image modality, comprise respective MRI datasets.

Also, the catheter guiding unit 707 may comprise a tool to enable a clinician to manipulate the catheter, using the displayed target region as a reference. It is not necessary that the catheter is guided automatically by the system.

In an embodiment, the catheter guiding unit 707 is configured to receive data indicative of the target region from the data processing unit 702. The catheter guiding unit 707 may comprise control means (not shown) to automatically guide the catheter towards the target region based on the registered target region. The data processing unit 702 may comprise a disease severity measurement determining unit 703 for determining a local disease severity measure based on a 3D dataset. The data processing unit 702 may further comprise a target region determining unit 704 for determining a target region for a treatment, based on the local disease severity measure wherein the target region corresponds to a transition region between a region with a low local disease severity measure and a region with a high local disease severity measure. The data processing unit 702 may further comprise a registering unit 705 for registering at least the target region with an interventional image modality.

The system may further comprise a guiding unit 707 for controlling an intramyocardial catheter guiding apparatus (not shown) to guide the catheter towards the target region based on the registered target region.

The system may further comprise means (not shown) to select a set of parameters to define a border zone of the diseased location. This may be implemented by means of a user interface.

It will be understood that the interventional imaging modality 706 and the catheter guiding unit 707 constitute a system for use in real-time image guided stem cell injection. The guiding unit 707 may be configured for controlling an intramyocardial catheter guiding apparatus to guide an intramyocardial catheter to a location on a target region corresponding to a border zone of a diseased area according to infarct transmurality, ischemia, or myocardial deformation.

It will be understood that the system may be implemented by means of a controller programmed by software to perform the steps disclosed in relation to the methods described herein, such as the method disclosed in relation to FIG. 6.

Some or all aspects of the invention may be suitable for being implemented in form of software, in particular a computer program product. Such computer program product may comprise a storage media on which the software is stored. Such a storage media may comprise, for example, an optical disc, magnetic disk, or flash memory. Also, the computer program may be represented by a signal, such as an optic signal or an electro-magnetic signal, carried by a transmission medium such as an optic fiber cable or the air. The computer program may partly or entirely have the form of source code, object code, or pseudo code, suitable for being executed by a computer system. For example, the code may be directly executable by one or more processors. Alternatively, the code may be interpreted by an interpreter that is executed by one or more processors. It will be understood that portions of the systems described herein may be implemented in form of software. Moreover, the method steps described herein may be implemented partially or completely in software. The software may be organized by means of subroutines. The subroutines may be combined to form a standalone executable program. Alternatively, the subroutines may be organized as a dynamically linkable library. A main program executable file may be provided that uses the subroutines from the dynamically linkable library. Each of the processing steps and/or system components described herein may be represented by executable code, be it in a dynamically linked library or in an executable file. Some, or all, of the functionality may be implemented as part of an operating system, some functionality may be implemented in a dynamically linked library, and some functionality may be implemented as an application program file.

The examples and embodiments described herein serve to illustrate rather than limit the invention. The person skilled in the art will be able to design alternative embodiments without departing from the scope of the claims. Reference signs placed in parentheses in the claims shall not be interpreted to limit the scope of the claims. Items described as separate entities in the claims or the description may be implemented as a single hardware or software item combining the features of the items described.

### REFERENCES

1. Dimmeler S, Zeiher A M, Schneider M D. Unchain my heart: the scientific foundations of cardiac repair. The Journal of Clinical Investigation. 2005; 115(3):572-83.
2. Orlic D, Kajstura J, Chimenti S, Bodine D M, Leri A, Anversa P. Bone marrow stem cells regenerate infarcted myocardium. Nature. 2003; 410(April):701-5.
3. Smits A M, Laake L W V, Ouden K D, Schreurs C, Szuhai K, Echteld C N, et al. Human cardiomyocyte progenitor cell transplantation preserves long-term function of the infarcted mouse myocardium. Cardiovascular Research. 2009; 83(3):527-35.
4. Beltrami A P, Barlucchi L, Torella D, Baker M, Limana F, Chimenti S, et al. Adult cardiac stem cells are multipotent and support myocardial regeneration. Cell. 2003; 114(6):763-76.
5. Loffredo F S, Steinhauser M L, Gannon J, Lee R T. Bone marrow-derived cell therapy stimulates endogenous cardiomyocyte progenitors and promotes cardiac repair. Cell stem cell. 2011; 8(4):389-98.
6. Quevedo H C, Hatzistergos K E, Oskouei B N, Feigenbaum G S, Rodriguez J E, Valdes D, et al. Allogeneic mesenchymal stem cells restore cardiac function in chronic ischemic cardiomyopathy via trilineage differentiating capacity. Proceedings of the national academy of science. 2009; 106(33):14022-7.
7. Duran J M, Taghavi S, Berretta R M, Makarewich C A, Sharp Iii T, Starosta T, et al. A characterization and targeting of the infarct border zone in a Swine model of myocardial infarction. Clinical and translational science. 2012; 5(5):416-21.
8. Ben-Haim S A, Osadchy D, Schuster I, Gepstein L, Hayam G, Josephson M E. Nonfluoroscopic, in vivo navigation and mapping technology. Nature Medicine. 1996; 2(12): 1393-5.
9. van Ramshorst J, Atsma D E, Beeres SLMA, Mollema S A, Ajmone Marsan N, Holman E R, et al. Effect of intramyocardial bone marrow cell injection on left ventricular dyssynchrony and global strain. Heart (British Cardiac Society). 2009; 95(2):119-24.
10. van Ramshorst J, Antoni M L, Beeres SLMA, Roes S D, Delgado V, Rodrigo S F, et al. Intramyocardial bone marrow-derived mononuclear cell injection for chronic myocardial ischemia: the effect on diastolic function. Circulation Cardiovascular imaging. 2011; 4(2):122-9.
11. Perin E C. Assessing Myocardial Viability and Infarct Transmurality With Left Ventricular Electromechanical Mapping in Patients With Stable Coronary Artery Disease: Validation by Delayed-Enhancement Magnetic Resonance Imaging. Circulation. 2002; 106(8):957-61.
12. Gyöngyösi M, Lang I, Dettke M, Beran G, Graf S, Sochor H, et al. Combined delivery approach of bone marrow mononuclear stem cells early and late after myocardial infarction: the MYSTAR prospective, randomized study. Nature clinical practice Cardiovascular medicine. 2009; 6(1):70-81.
13. Gyöngyösi M, Dib N. Diagnostic and prognostic value of 3D NOGA mapping in ischemic heart disease. Nature Reviews Cardiology. 2011; 8(7):393-404.
14. van Ramshorst J, Bax J J, Beeres SLMA, Dibbets-Schneider P, Roes S D, Stokkel M P M, et al. Intramyocardial bone marrow cell injection for chronic myocardial ischemia: a randomized controlled trial. JAMA: the journal of the American Medical Association. 2009; 301(19): 1997-2004.
15. van der Spoel T I G, Vrijsen K R, Koudstaal S, Sluijter J P G, Nijsen J F W, de Jong H W, et al. Transendocardial cell injection is not superior to intracoronary infusion in a porcine model of ischaemic cardiomyopathy: a study on delivery efficiency. Journal of cellular and molecular medicine. 2012; 16(11):2768-76.
16. Heiberg E, Sjögren J, Ugander M, Carlsson M, Engblom H, Arheden H. Design and validation of Segment—freely available software for cardiovascular image analysis. BMC medical imaging. 2010; 10(1):1-13.
17. Heiberg E, Engblom H, Engvall J, Hedström E, Ugander M, Arheden H. Semi-automatic quantification of myocardial infarction from delayed contrast enhanced magnetic resonance imaging. Scandinavian cardiovascular journal: SCJ. 2005; 39(5):267-75.
18. Arun K S, Huang T S, Blostein S D. Least squares fitting of two 3D point sets. IEEE Transactions on Pattern Analysis and Machine intelligence. 1987; PAMI-9(5):698-700.
19. Umeyama S. Least-squares estimation of transformation parameters between two point patterns. IEEE Transactions on Pattern Analysis and Machine intelligence. 1991 1991; 13(4):376-80.
20. Wijnmaalen A P, Geest RJVD, Van Huls Van Taxis C F B, Siebelink H-mJ, Kroft L J M, Bax J J, et al. Head-to-head comparison of contrast-enhanced magnetic resonance imaging and electroanatomical voltage mapping to assess post-infarct scar characteristics in patients with ventricular tachycardias: real-time image integration and reversed registration. European Heart Journal. 2010; 32(1):104-14.

1. A method of processing an image, comprising
determining a local disease severity measure based on a previously obtained 3D volume dataset;
determining a target region for a treatment, based on the local disease severity measure, wherein the target region corresponds to a transition region between a first region and a second region, wherein the first region has values of the local disease severity measure which are substantially distinct from the values of the local disease severity measure in the second region; and
registering at least the target region with an interventional image modality.

2. The method of claim 1, wherein said local disease severity measure is indicative of a measure of a local disease severity at a location along a surface of a myocardium.

3. The method of claim 1, wherein the local disease severity measure is indicative of a transmurality of an infarction of a myocardium or of a local severity of ischemia.

4. The method of claim 3, wherein the transition zone is a border zone of an infarcted region of the myocardium.

5. The method of claim 1, wherein said determining the target region comprises comparing the local disease severity measure to a reference value.

6. The method of claim 5, wherein the target region comprises a region where the local disease severity is equal to the reference value, a region where the local disease severity is smaller than the reference value, and a region where the local disease severity is larger than the reference value.

7. The method of claim 4, wherein the target region corresponds to a region comprising an isoline where the local disease severity is equal to the reference value and a margin on both sides of the isoline.

8. The method of claim 6, wherein the margin has a predetermined width measured along a surface of the myocardium, on both sides of the isoline.

9. The method according to claim 1, further comprising determining a no-go zone for intervention corresponding to an area with a high local disease measure according to a set of predetermined constraints.

10. The method of claim 1, wherein the 3D dataset comprises one or a combination of a nuclear magnetic resonance dataset, a magnetic resonance imaging dataset, a late gadolinium enhanced magnetic resonance imaging dataset, an endogenous contrast MRI dataset wherein no contrast materials are used, a myocardial deformation MRI dataset, a computed tomography dataset, a PET dataset, a SPECT dataset, an echography dataset, and a spectroscopy dataset.

11. The method of claim 1, wherein the interventional image modality comprises an interventional navigation system.

12. The method of claim 1, wherein the interventional image modality comprises a magnetic resonance imaging modality, wherein the method comprises acquiring an interventional magnetic resonance imaging dataset while navigating the catheter inside a body, and wherein the step of registering comprises registering the target region with the interventional magnetic resonance imaging dataset.

13. The method of claim 1, further comprising displaying the registered target region fused with an image of the interventional image modality during an intervention, or displaying the registered target region in a bull's eye plot format or a polar coordinate system.

14. The method of claim 1, further comprising directing a catheter onto the target region.

15. The method of claim 14, further comprising injecting at least one stem cell, or stem cell derived factors, or medication, or biomaterials, or a combination into the target region using the catheter.

16. The method of claim 14, further comprising performing an ablation or performing a biopsy in the target area using the catheter.

17. A system for processing an image, comprising
a disease severity measurement determining unit for determining a local disease severity measure based on a 3D dataset;
a target region determining unit for determining a target region for a treatment, based on the local disease severity measure wherein the target region corresponds to a transition region between a region with low values of the local disease severity measure and a region with a high values of the local disease severity measure, wherein the low values are lower than the high values of the local disease severity measure; and
a registering unit for registering at least the target region with an interventional image modality.

18. The system of claim 17, further comprising a guiding unit for controlling an intramyocardial catheter guiding apparatus to guide the catheter towards the target region based on the registered target region.

19. The system as described in claim 17, comprising means to specify the definition of border zone of the diseased location.

20. A system for use in real-time image guided stem cell injection, the system comprising a guiding unit for controlling an intramyocardial catheter guiding apparatus to guide an intramyocardial catheter to a location on a target region corresponding to a border zone of a diseased area according to infarct transmurality, ischemia, or myocardial deformation.
